(19) United States
(12) Patent Application Publication
(10) Pub. No.: US 2009/0209992 A1
(43) Pub. Date: Aug. 20, 2009

(54) DISPOSABLE STERILIZED TATTOO MACHINE

(76) Inventor: David McConchie, Dearborn, MI (US)

Correspondence Address: ITALIA IP, 3500 WEST OLIVE AVE., SUITE 300, BURBANK, CA, 91505, US

(21) Appl. No.: 12/367,478

(22) Filed: Feb. 6, 2009

Related U.S. Application Data
(60) Provisional application No. 61/029,451, filed on Feb. 18, 2008.

(51) Int. Cl. A61B 17/34; B65B 43/26

(52) U.S. Cl. 606/186; 53/492

(57) ABSTRACT

A disposable sterilized tattoo machine comprising a pre-assembled tattoo machine which is sterilized as an assembled tattoo machine and stored and/or sold in sterilized package prior to use and disposed of as an assembled tattoo machine after use on a client. The sterilized tattoo machine according to the present invention includes a motor mounted on a housing that includes an opening to receive a commercially available needle tube, and an electrical connector by which the tattoo machine may be connected for power and disconnected prior to disposal. The needle tube is located relative to the motor to permit the end of the needle to resiliently remain on the connecting element (e.g. eccentric pin) of the motor when assembled, yet allow for easy assembly, adjustment and removal of the needle and needle tube.

### CROSS-REFERENCE TO RELATED APPLICATION

This application claims the benefit of the filing date under 35 USC 119(e) of the filing date of U.S. Provisional Application Ser. No. 61/029,451, filed Feb. 18, 2008, the contents of which are incorporated herein by reference.

### FIELD OF THE INVENTION

This application relates generally to tattoo machines, and in particular to disposable tattoo machines and tattoo machine housings.

### BACKGROUND

The typical tattoo process typically involves user operation of a somewhat bulky tattoo “machine” that comprises an assembly of various components, a needle extending through a tube that contains or receives colored inks, and a motor which imparts a reciprocating motion on the needle, and a housing or holder of these elements.

Tattoo equipment and the tattooing procedures need to be sterile, and to that end, the tattoo machine should be sterilized and sterizable. However, previous tattoo machines and equipment are typically assembled, disassembled and repeatedly re-assembled from individual components that can be complex to assemble and disassembled for sterilization.

### SUMMARY

The disposable sterilized tattoo machine according to the present invention comprises a pre-assembled tattoo machine which is sterilized as an assembled tattoo machine and stored and/or sold in sterilized package prior to use and disposed of as an assembled tattoo machine after use on a client. The sterilized tattoo machine according to the present invention includes a motor mounted on a housing that includes an opening to receive a commercially available needle tube, and an electrical connector by which the tattoo machine may be connected for power and disconnected prior to disposal. The needle tube is located relative to the motor to permit the end of the needle to resiliently remain on the connecting element (e.g. eccentric pin) of the motor when assembled, yet allow for easy assembly and adjustment of the needle and needle tube.

### BRIEF DESCRIPTION OF DRAWINGS

The drawings, when considered in connection with the following description, are presented for the purpose of facilitating an understanding of the subject matter sought to be protected.

FIG. 1 is a perspective view of one embodiment according to the present invention;

FIG. 2 is an alternate perspective view of the housing according to the embodiment of FIG. 1; and

FIG. 3 is a flowchart of an exemplary process of preparing and using an apparatus according to one embodiment of the present invention.

### DESCRIPTION OF EXEMPLARY EMBODIMENT

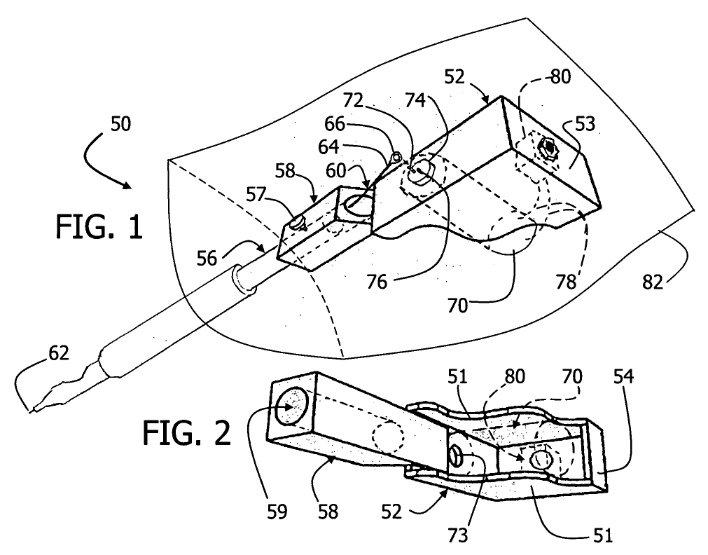

The embodiment 50 shown in FIG. 1 includes four basic parts, i.e. a motor housing assembly 52, a motor flywheel 72 attached to a motor 70 a flexible shaft and needle assembly 56, and a needle tube holder 58. The motor housing 52 and/or the needle tube holder 58 typically comprises part injection molded from a medical grade plastic material such as polypropylene, but may also be fashioned from another molded or machined material, e.g. stainless steel, etc. The motor 70 typically comprises a commercially available model, or can be custom made to achieve the reciprocating motion required by the invention.

The needle tube 56 typically comprises a commercially available needle tube, or custom made to contain or receive ink as may be appropriate for the tattoo, and include a passage therein to receive a flexible needle shaft 60 therethrough. While one end 62 emerges from the tip of the needle tube 56, the opposite end 64 of the needle is retained by an eyelet 66 having an aperture therein. The needle tube holder 58 includes a tubular aperture 59 dimensioned to receive the particular needle tube 56 used which is retained by a needle tube holder and adjuster 57 typically comprising a screw threaded into the holder 58 until it engages the needle tube 56 disposed within the aperture 59. The aperture 59 extends through the needle tube holder 58 along an axis which is generally parallel to the needle when disposed in the needle tube and connected to the motor flywheel 74. Typically the flexible shaft 60 comprises a medical grade stainless steel wire and the eyelet 66 comprises a brass eyelet that retains the end 64, which wire is cut to the appropriate length, wrapped around the eyelet 66 having an aperture therein, and which is deformed (crimped) to secure the needle shaft end 64 to the eyelet. Needles may also be selected and attached (e.g. by soldering) to the end 62 of the flexible shaft 60 by any acceptable method.

The flywheel 72 includes an eccentric 74, such as a pin radially displaced from the motor 70 shaft 74 axis. The motor 70 is retained by the motor housing assembly 52 typically with screws extending therethrough into the motor 70, and the housing assembly 52 has an aperture 73 (FIG. 2) through which the shaft 76 passes and the flywheel 72 is attached (e.g. press-fit thereon). The motor housing 52 provides a planar surface offset from the axis of the needle flexible shaft 60 and on which motor 70 is mounted such that dimension of the flywheel 72 may be accommodated and allow the eccentric 74 to engage the eyelet 66 aperture substantially along the undeflected axis of the flexible shaft 66 when inserted into the needle tube 56. By applying a force generally perpendicular to the axis of the flexible shaft 60 through the needle tube, the eyelet 66 may be disengaged from the flywheel 72 eccentric 74 as shown in FIG. 1; otherwise, the alignment of the axis of the flexible shaft 60 and the flywheel 72 eccentric 74 generally urges the eyelet 66 to remain on the eccentric 74.

The motor 70 is removably connected external power via a connector 80 such as an ⅛″ jack, mounted on a flange 53 typically extending perpendicular to the motor housing 52 surface and via wires 78 connecting the motor 70 and the connector 80. When the motor 70 is energized, the flywheel rotates causing the eccentric 74 to move in an orbital motion imparting a reciprocating motion on the needle flexible shaft 60. Alternate motors are also included which connect to and impart a reciprocating motion to the needle flexible shaft 60.

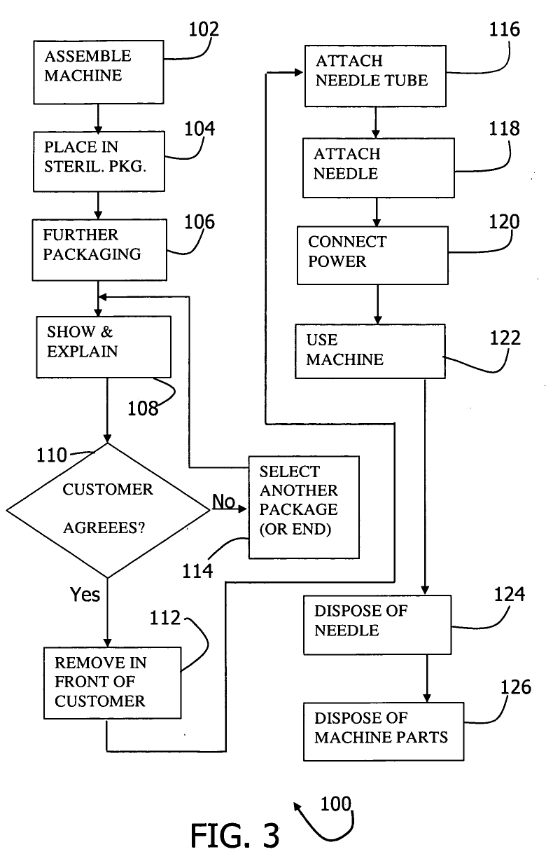

The preparation and use of an exemplary embodiment according to the present invention is shown in the flowchart 100 of FIG. 3, wherein the embodiment is assembled 102 and is then placed 104 in a sterilizable package (82 of FIG. 1) which when sealed provide a verifiable container for the sterilized tattoo machine, with a set of flexible shaft needles, sealed and place in a sterilization chamber for sterilization. The resulting sterilized units (in the package) are further packaged 106 into another shipping container for protection and shipping to end user. Upon receipt, the sterilized embodiment is shown to the customer and explained 108 how to determine whether the packaged assembled tattoo machine is sterilized. If the customer agrees 110 that it is sterilized or the sterilized machine, the user will remove 112 the contents from the sterilized package in front of the tattoo customer to ensure that the agreed upon unit is in fact being used. If the customer does not agree, another package is selected 114 or the process is ended. The user will then attach 116 the appropriate sterilized needle tube to the sterilized disposable tattoo machine and add 118 the appropriate flexible shaft/needle, which is slid down through the tube 56 and holder 58 and attached to the flywheel of the motor by simply sliding the brass eyelet onto the offset eccentric 74 shaft of the flywheel 72. The user will then attach the power source by plugging 120 a power plug into the jack 80 located on the flange 53, and the tattoo machine is now ready for use 122. When the user completes the work for each sitting, the flexible needle shafts are removed from the tattoo machine and discarded 124 using the current process for disposal of needles. The remaining tattoo machine 50 parts will be disposed of 126 according to the current practices for the needles.

An alternate view of the embodiment of the motor housing 52 and the needle tube holder 58 is shown in FIG. 2, and are connected together by welding (chemically, ultrasonically or thermally), bonded by glue or adhesive, riveted, screwed, interlocked or otherwise permanently or removably connected, or formed as a single unit. Also shown are reinforcement sidewalls 51 and bottom member 54 connected to the sidewalls 51 and flange 53. The motor 70 is shown relative to the aperture 73 and the connector 80.

These and further modifications and substitutions made by one of ordinary skill in the art are within the scope of the present invention which is not to be limited except by the claims which follow.

What is claimed is:

1. A tattoo machine, comprising:
a motor housing;
a needle tube holder contiguous with said motor housing and connected thereto;
a motor retained by said motor housing, said motor further including a flywheel; and
a needle assembly including a needle comprising a flexible shaft having a first end received through said needle tube holder and connector disposed on a second end of said needle, said connector movably connected to said flywheel to impart a reciprocating motion by action of said motor.

2. The tattoo machine of claim 1, wherein said flywheel includes and eccentric disposed to provide motion about an axis, and
said needle is connected to an eyelet having said second end wrapped thereabout, said eyelet having an aperture received over an eccentric disposed on said flywheel to impart said reciprocating motion to said needle.

3. The tattoo machine of claim 1, further including a motor power connector disposed on said motor housing.

4. The tattoo machine of claim 1, comprising sterilizable components.

5. The tattoo machine of claim 4, further including an enclosure providing a sterilized environment therein.

6. A tattoo machine housing, comprising
a tubular member having a longitudinal aperture extending along an axis and a cross-section substantially perpendicular to said axis substantially smaller than the length of said longitudinal aperture;
a planar member having a surface connected to said tubular member, substantially parallel to and offset from said tubular member axis, and further having a length dimension and an aperture along said length at a distance from said tubular member; and
a flange extending disposed substantially perpendicular to planar member distal from said tubular member and offset and substantially perpendicular to said axis.

7. The tattoo machine housing of claim 6, wherein said flange includes a connector aperture thereon.

8. The tattoo machine housing of claim 6, wherein said planar member and said tubular member are connected by at least one of glue, a weld, a crimp, a rivet, and a screw.

9. The tattoo machine housing of claim 6, wherein said planar member and said flange member formed from single element comprising said motor housing.

10. The tattoo machine housing of claim 6, wherein said tubular member is adapted to receive needle tube therethrough.

11. The tattoo machine housing of claim 6 comprising sterilizable material.

12. A method of sterile tattooing, comprising the steps of:
storing said sterilized tattoo machine and at least one needle in a verifiable package;
sterilizing an assembled tattoo machine together with at least one needle;
opening said verifiable package and revealing said sterilized tattoo machine and said at least one said sterilized needle; and
customer approving of the use of said sterilized tattoo machine and said at least one sterilized needle.

13. The method of claim 12, further including the step of inserting a sterilized needle into said sterilized tattoo machine.

14. The method of claim 13, further including the step of disposing of said tattoo machine according to acceptable practices for disposing tattoo needles.
